(19) United States
(12) Patent Application Publication
(10) Pub. No.: US 2005/0170004 A1
(43) Pub. Date: Aug. 4, 2005

(54) Nanoparticles for drug delivery

(76) Inventors: Vered Rosenberger, Jerusalem (IL); Naiomi Moldavski, Jerusalem (IL); Moshe Flashner-Barak, Petach Tikva (IL); E. Itzhak Lerner, Petach Tikva (IL)

Correspondence Address: COOPER & DUNHAM, LLP, 1185 AVENUE OF THE AMERICAS, NEW YORK, NY, 10036, US

(21) Appl. No.: 10/977,926

(22) Filed: Oct. 28, 2004

Related U.S. Application Data
(60) Provisional application No. 60/516,324, filed on Oct. 31, 2003.

(51) Int. Cl. A61K 9/16; A61K 9/50

(52) U.S. Cl. 424/490

(57) ABSTRACT

A pharmaceutical composition comprising a nanoparticle and any one of a peptide, a polysaccharide, or a glycoprotein, attached electrostatically thereto, and a pharmaceutically acceptable carrier. Uses of the composition and processes for its preparation.

This application claims the benefit of U.S. Provisional Application No. 60/516,324, filed Oct. 31, 2003, the contents of which are hereby incorporated by reference.

Throughout this application various publications are referenced in parenthesis. The disclosures of these publications in their entireties are hereby incorporated by reference into this application in order to more fully describe the state of the art to which this invention pertains.

### BACKGROUND OF THE INVENTION

Protein and peptide drugs are becoming more common as the fruits of biotechnology become available. Such drugs suffer from the necessity of delivering them by injection. Oral delivery is precluded by both the lack of oral bioavailability and by the efficient degradation of such molecules by the enzymatic systems of the gastrointestinal (GI) tract. Peptides and proteins are large, usually hydrophilic, molecules. Hydrophilic molecules are poorly absorbed by passive diffusion. The cell walls of the intestine are permeable to hydrophobic molecules but not to hydrophilic molecules. The junctions between the cells are closed tightly (therefore they are called the “tight junctions”) and are permeable only to relatively small hydrophilic molecules. The ileal region of the small intestine has certain specialized cells (M cells and Peyer's patches) that allow entry of large molecules. However, these cells are more efficient at ingesting particles than solutions. Another part of the oral-gastrointestinal tract is the sublingual region of the oral cavity. Sublingual absorption of drugs in solution is found for hydrophobic drugs such as nitroglycerin but the sublingual membranes are not permeable to hydrophilic compounds.

The problem of oral delivery for protein and peptide drugs has been addressed by many research groups. One offered solution is the use of microemulsions and/or of nanoparticles. Microemulsions are defined as emulsions of oil in water wherein the average particle size of the oil droplet is in the low hundreds of nanometers range (e.g. <200 nm). Microemulsions can be formed by emulsifying oil in water with surfactants with a proper input of energy such as high speed stirring, ultrasound irradiation or high pressure filtration. Some oil or wax formulations with nonionic surfactants spontaneously form microemulsions when gently mixed with water. Microemulsions may incorporate relatively hydrophobic peptides or proteins and offer a way of delivering them. When the peptide is incorporated within the oil droplet it should be protected, at least partially, from enzymatic degradation.

However, microemulsions cannot be a general method for delivering peptide drugs for several reasons. Most peptides are hydrophilic and will not be incorporated into the oil droplets of the emulsion but rather will reside in the water phase. Thus, the peptide or protein will not be protected against degradation. Also, emulsion droplets (water phase) do not offer any particular mechanism for the drug to be absorbed into the lumen of the GI tract, and the droplets in an emulsion tend to be labile, changing in size etc. as droplets merge and split up.

Nanoparticles can be made by incorporating a solute, usually a polymer, within the oil phase of a microemulsion of solvent in water. By using double emulsion techniques (water/oil/water) one can incorporate hydrophilic molecules, such as most peptides, within the nanoparticle. Removal of the solvent by evaporation or extraction leaves the peptide incorporated in the nanoparticle. Peptides incorporated within such nanoparticles have been shown to be protected against enzymatic degradation (Lowe, P. J., Temple, C. S., Calcitonin and Insulin in isobutylcyanoacrylate Nanocapsules: Protection against Proteases and Effect on Intestinal Absorption in Rats, J. Pharm. Pharmacol. (1994) 46(7), 547-552; Almeida, A. J., Runge, S., Muller, R. H., Peptide-loaded Solid Lipid Nanospheres, Int. J. Pharm. (1997), 149(2), 255-265) and may be available for delivery through Peyer's patches and the like. However, such nanoparticles tend to be of relatively slow releasing nature. This property has been used as an advantage for injectable depots but may be a detriment to the availability of the drug when delivered orally. Peptides have also been incorporated among hydrophilic chains of polymers coated on a hydrophobic nanoparticle (Sakuma, S., et. al., Stabilization of Salmon Calcitonin by Polystyrene nanoparticles having Surface Hydrophilic polymeric Chains, Against Enzymatic Degradation, Int. J. Pharm. (1997), 159(2), 181-189).

An elegant method for forming nanoparticles has been developed by Mumper and co-workers who take wax/surfactant mixtures that form spontaneous microemulsions when in the melt state at above 50° C. and then cool to room temperature to form nanoparticles. Radionucleotides (Oyewumi, M. O., Mumper, R. J. Engineering Tumor Targeted Gadolinium Hexanedione Nanoparticles for Potential Application in Neutron Capture Therapy, Bioconjug. Chem. (2002), 13(6), 1328-35; Oyewumi, M. O., Mumper, R. J, Gadolinium Loaded Nanoparticles Engineered from Microemulsion Templates, Drug Dev. Ind. Pharm. (2002), 28(3), 317-28) have been incorporated into the nanoparticles. In addition, by incorporating the cationic surfactant cetyltrimethylammonium bromide into the molten microemulsion and subsequent solidifying to nanoparticles they were able to bind DNA to the outside of the nanoparticles (Cui, Z., Mumper, R. J., Genetic Immunization using Nanoparticles Engineered from Microemulsion Precursors, Pharm. Res. (2002), 19(7), 939-46) and were able to bind cationized proteins (proteins specially treated to add cationic groups to the protein surface) when using the anionic surfactant sodium lauryl sulfate (Cui, Z. Mumper, R. J., Coating of Cationized Protein on Engineered Nanoparticles Results in Enhanced Immune Responses, Int. J. Pharm., (2002), 238(1-2), 229-39). The protein was cationized to make the protein bind to the surface of the anionic nanoparticle by electrostatic forces. There is no indication that this method would work for non-cationized proteins and surely not for peptides that are shorter than proteins. Mumper's work, directed towards immunization, also implies that the proteins or DNA so attached to the outside of the particle are available for recognition and, similarly, available for degradation.

While investigating this mode of binding to charged nanoparticles formed from the cooling of microemulsions spontaneously formed above room temperature we have surprisingly found that nanoparticles formed from microemulsions that incorporate charged surface active agents are capable of binding non modified peptides to their surface. Further, while the down side of such a system was expected to be a lack of protection of the drug from degradative processes, we have also surprisingly found that the interaction with the nanoparticles also confers stability on the peptide towards enzymatic degradation while maintaining drug presentation. This phenomenon should be helpful for all forms of delivery of the peptide whether through the ileum, intra venous, subcutaneous, intra-arterial or intramuscular injections. We have further surprisingly found that small diameter nanoparticles of this nature can be absorbed through the sublingual membrane offering another mode of drug delivery.

### SUMMARY OF THE INVENTION

This invention provides a pharmaceutical composition comprising a nanoparticle and any one of a peptide, a polysaccharide, or a glycoprotein, attached electrostatically thereto, and a pharmaceutically acceptable carrier.

An embodiment of the invention is a pharmaceutical composition comprising nanoparticles of i) an admixture of mono-, di-, and tri-glycerides with free polyethylene glycol and with mono-, and di-fatty acid esters of polyethylene glycol, ii) sodium docusate, and iii) glatiramer acetate.

Also provided is a process for preparing the pharmaceutical compositions described comprising i) forming a spontaneous microemulsion by heating to above 50° C. a mixture of water, and a wax; ii) cooling the microemulsion to room temperature to form nanoparticles; and iii) contacting the nanoparticles with the peptide, the polysaccharide, or the glycoprotein to form the pharmaceutical composition.

Also provided by this invention is a method of delivering to a subject a peptide, a polysaccharide, or a glycoprotein, comprising administering to the subject the pharmaceutical composition described here. The administration may be sublingual, orally to the stomach, orally to the small intestine, orally to the large intestine, intramuscular, subcutaneous, intra-arterial or intravenous.

Thus, also provided herein is a general method of inhibiting enzymatic degradation of a peptide, a polysaccharide, or a glycoprotein upon oral ingestion of the peptide, the polysaccharide, or the glycoprotein by an animal, comprising electrostatically attaching the peptide, the polysaccharide, or the glycoprotein to a nanoparticle prior to the oral ingestion, so as to thereby inhibit enzymatic degradation of the peptide, the polysaccharide, or the glycoprotein upon oral ingestion.

Yet furthermore, this invention provides a method of delivering to a subject a deoxyribonucleic acid molecule or a ribonucleic acid molecule, comprising administering to the subject a pharmaceutical composition comprising the deoxyribonucleic acid molecule or the ribonucleic acid molecule attached electrostatically to a nanoparticle, and a pharmaceutically acceptable carrier, wherein the administration is oral or sublingual. In this method the nanoparticle may comprises an organic wax having a melting point from 40° C. to 60° C., as described previously. However, if any ionic surfactant is present, it is a cationic surfactant, such as, for example, cetyltrimethylammonium bromide, chlorhexidine salts, hexadecyl triammonium bromide, dodecyl ammonium chloride or an alkylpyridinium salt.

### BRIEF DESCRIPTION OF THE FIGURES

FIG. 1. Daily mean scores of each treatment group in the rat Experimental autoimmune encephalomyelitis (EAE) Model.

FIG. 2. Kinetics of IL-2 secretion from spleen cells of mice orally treated with glatiramer acetate reference standard (GA RS) and glatiramer acetate (GA) formulations 3, 6 and 10 days of feeding.

FIG. 3. Kinetics of TGF-beta secretion from spleen cells of mice orally treated with GA RS and GA formulations 3, 6 and 10 days of feeding.

### DETAILED DESCRIPTION OF THE INVENTION

This invention provides a pharmaceutical composition comprising a nanoparticle and any one of a peptide, a polysaccharide, or a glycoprotein, attached electrostatically thereto, and a pharmaceutically acceptable carrier.

The nanoparticle may comprise an organic wax having a melting point from 40° C. to 60° C. The organic wax may be stearic acid; atomized glyceryl palmitostearate; atomized glyceryl behenate; a paraffin wax having a melting point from 40° C. to 60° C.; a mixture of mono-, di-, and tri-glycerides obtained by esterification of fatty acids of natural origin with glycerol and having a melting point from 40° C. to 60° C.; a mixture of mono-, di-, and tri-glycerides obtained by transesterification of fatty acids of natural origin and having a melting point from 40° C. to 60° C.; or a mixture of mono-, di-, and tri-glycerides of C12-C18 fatty acids and having a melting point from 40° C. to 60° C.

The organic wax may be admixed with a nonionic surfactant. The admixture of organic wax and non-ionic surfactant may be cetyl alcohol with polysorbate 60, polyoxyl 2 stearyl ether with polysorbate 80, or mono-, di-, and tri-glycerides with free polyethylene glycol and with mono-, and di-fatty acid esters of polyethylene glycol.

The nanoparticle can comprise an ionic surface-active agent. The surface-active agent may have a charged head and a hydrophobic tail, and can be an anionic surfactant, such as, for example, sodium lauryl sulfate, sodium cholate, sodium taurocholate, or sodium docusate. In one embodiment, the surface-active agent is sodium docusate.

In one embodiment, the peptide, the polysaccharide, or the glycoprotein has a net positive charge and the nanoparticle has a net negative charge, such that the peptide, the polysaccharide, or the glycoprotein is electrostatically attached to the nanoparticle. In such an embodiment, the peptide attached to the nanoparticle may have more combined total lysine plus arginine groups than the combined total of aspartic acid plus glutamic acid groups. In such an embodiment, the peptide may be glatiramer acetate or an interferon; and the polysaccharide may be gentamycin, amikacin or tobramysin.

In another embodiment, the surface-active agent may be a cationic surfactant, such as, for example, cetyltrimethylammonium bromide, chlorhexidine salts, hexadecyl triammonium bromide, dodecyl ammonium chloride or an alkylpyridinium salt.

In an embodiment, the peptide, the polysaccharide, or the glycoprotein may have a net negative charge and the nanoparticle has a net positive charge, such that the peptide, the polysacharide, or the glycoprotein is electrostatically attached to the nanoparticle. In such an embodiment, the peptide attached to the nanoparticle can has more combined total aspartic acid plus glutamic acid groups than lysine plus arginine groups. In such an embodiment, the polysaccharide attached to the nanoparticle may be heparin.

In any embodiment of the invention, the pharmaceutical composition may be characterized in that the rate of enzymatic degradation of the peptide, the polysaccharide, or the glycoprotein when electrostatically attached to the nanoparticle is lower than the rate of enzymatic degradation of the peptide, the polysaccharide, or the glycoprotein when unattached to the nanoparticle in solution.

Yet a further embodiment of the invention is a pharmaceutical composition comprising nanoparticles of i) an admixture of mono-, di-, and tri-glycerides with free polyethylene glycol and with mono-, and di-fatty acid esters of polyethylene glycol, ii) sodium docusate, and iii) glatiramer acetate.

In this embodiment, the admixture may comprise 60-90% by weight of the composition, wherein the sodium docusate comprises 2-30% by weight of the composition, and wherein the glatiramer acetate comprises 3-20% of the composition. Alternatively, in this embodiment, the admixture may comprise 80-85% by weight of the composition, wherein the sodium docusate comprises 5-7% by weight of the composition, and wherein the glatiramer acetate comprises 8-15% of the composition.

The admixture may comprise 20% mono-, di-, and tri-glycerides, 8% free polyethylene glycol, and 72% mono-, and di-fatty acid esters of polyethylene glycol.

In any of the embodiments of this invention, the pharmaceutical composition may be characterized in that the rate of enzymatic degradation of the glatiramer acetate when electrostatically attached to the nanoparticle is lower than the rate of enzymatic degradation of the glatiramer acetate when unattached to the nanoparticle in solution.

In any of the embodiments of this invention, the nanoparticle can have an average diameter of between 1 nm and 5000 nm. In certain embodiments, the nanoparticle has an average diameter of between 200 nm and 3000 nm; or an average diameter of between 500=m and 2000 nm; or an average diameter of between 1 nm and 1000 nm; or an average diameter of between 1 nm and 500 nm; or an average diameter of between 10 nm and 300 nm; or an average diameter of between 20 nm and 200 nm; or an average diameter of between 20 nm and 150 nm; or an average diameter of between 100 nm and 600 nm; or an average diameter of between 200 nm and 500 nm.

Another embodiment of this invention is a lyophilized pharmaceutical composition of any composition described here.

Also provided is a process for preparing the pharmaceutical compositions described comprising i) forming a spontaneous microemulsion by heating to above 50° C. a mixture of water, and a wax; ii) cooling the microemulsion to room temperature to form nanoparticles; and iii) contacting the nanoparticles with the peptide, the polysaccharide, or the glycoprotein to form the pharmaceutical composition.

In the process, the wax may be an organic wax having a melting point from 40° C. to 60° C., such as, for example, stearic acid; atomized glyceryl palmitostearate; atomized glyceryl behenate; paraffin waxes having a melting point from 40° C. to 60° C.; a mixture of mono-, di-, and tri-glycerides obtained by esterification of fatty acids of natural origin with glycerol and having a melting point from 40° C. to 60° C.; a mixture of mono-, di-, and tri-glycerides obtained by transesterification of fatty acids of natural origin and having a melting point from 40° C. to 60° C.; or a mixture of mono-, di-, and tri-glycerides of C12-C18 fatty acids and having a melting point from 40° C. to 60° C.

As in the composition, in the process, the wax may comprise a nonionic surfactant, or an ionic surfactant. The wax with non-ionic surfactant may be cetyl alcohol with polysorbate 60, polyoxyl 2 stearyl ether with polysorbate 80, or mono-, di-, and tri-glycerides with free polyethylene glycol and with mono-, and di-fatty acid esters of polyethylene glycol.

The ionic surfactant may be an anionic surfactant, such as, for example, sodium lauryl sulfate, sodium cholate, sodium taurocholate, or sodium docusate.

The ionic surfactant may also be a cationic surfactant, such as, for example, cetyltrimethylammonium bromide, chlorhexidine salts, hexadecyl triammonium bromide, dodecyl ammonium chloride or an alkylpyridinium salt.

In the process, the peptide, the polysaccharide, or the glycoprotein are as described for the composition.

Also provided by this invention is a method of delivering to a subject a peptide, a polysaccharide, or a glycoprotein, comprising administering to the subject the pharmaceutical composition described here. The administration may be sublingual, orally to the stomach, orally to the small intestine, orally to the large intestine, intramuscular, subcutaneous, intra-arterial or intravenous.

Also provided herein is a general method of inhibiting enzymatic degradation of a peptide, a polysaccharide, or a glycoprotein upon oral ingestion of the peptide, the polysaccharide, or the glycoprotein by an animal, comprising electrostatically attaching the peptide, the polysaccharide, or the glycoprotein to a nanoparticle prior to the oral ingestion, so as to thereby inhibit enzymatic degradation of the peptide, the polysaccharide, or the glycoprotein upon oral ingestion. The nanoparticle composition, its formation and attachment to the peptide, the polysaccharide and the glycoprotein are as described herein. For example, in one embodiment, the peptide may be glatiramer acetate; in another embodiment, the nanoparticle may comprise i) an admixture of mono-, di-, and tri-glycerides with free polyethylene glycol and with mono- and di-fatty acid esters of polyethylene glycol, and ii) sodium docusate; and the peptide, the polysaccharide, or the glycoprotein may be attached to the nanoparticle by i) forming a spontaneous microemulsion by heating to above 50° C. a mixture of water, and a wax; ii) cooling the microemulsion to room temperature to form nanoparticles; and iii) contacting the nanoparticles with the peptide, the polysaccharide, or the glycoprotein, thereby electrostatically attaching the peptide, the polysaccharide, or the glycoprotein to the nanoparticle.

Yet furthermore, this invention provides a method of delivering to a subject a deoxyribonucleic acid molecule or a ribonucleic acid molecule, comprising administering to the subject a pharmaceutical composition comprising the deoxyribonucleic acid molecule or the ribonucleic acid molecule attached electrostatically to a nanoparticle, and a pharmaceutically acceptable carrier, wherein the administration is oral or sublingual. In this method the nanoparticle may comprises an organic wax having a melting point from 40° C. to 60° C., as described previously. However, if any ionic surfactant is present, it is a cationic surfactant, such as, for example, cetyltrimethylammonium bromide, chlorhexidine salts, hexadecyl triammonium bromide, dodecyl ammonium chloride or an alkylpyridinium salt.

The subject invention provides a pharmaceutical composition prepared by any one of the disclosed processes.

The subject invention also provides any one of the disclosed pharmaceutical compositions comprising glatiramer acetate in an amount effective to treat an autoimmune disease or an inflammatory non-autoimmune disease in a subject, and a pharmaceutically acceptable carrier.

In one embodiment, the autoimmune disease is multiple sclerosis.

The subject invention also provides a method for treating a subject afflicted with an autoimmune disease or an inflammatory non-autoimmune disease, which comprises administering to the subject any of the disclosed pharmaceutical compositions.

In another embodiment, the administration is through intravenous, intraperitoneal, intramuscular, subcutaneous, oral, intranasal, buccal, vaginal, rectal, intraocular, intrathecal, topical, sublingual or intradermal routes.

In a further embodiment, the administration is oral.

The subject invention further provides a method of treating a subject afflicted with relapsing remitting multiple sclerosis which comprises oral administration of a nanoparticulate formulation of glatiramer acetate, wherein the amount of glatiramer acetate in the nanoparticulate formulation is effective to alleviate a symptom of the relapsing-remitting multiple sclerosis in the subject.

Definitions

The term “nanoparticle” as used in this document refers to a particle having an average size of 1-5000 nanometers (nm). The chemical composition of the nanoparticle can vary as described throughout this document.

The terms “peptide” as used in this document refers to a peptide or a protein whose net electric charge, if any, is due solely to its composition of naturally occurring amino acids, and whose net electric charge has not been modified by the covalent addition of a non-amino acid molecule. The term “peptide” as used in this document includes within its definition a protein.

The term “polysaccharide” as used in this document refers to a polysaccharide whose net electric charge, if any, is due solely to its composition of naturally occurring sugars.

The term “glycoprotein” as used in this document refers to a glycoprotein whose net electric charge, if any, is due solely to its composition of naturally occurring amino acids and sugars.

Production of Nanoparticles

Nanoparticles can be produced by milling of macro or microparticles in special mills of high energy to obtain nanoparticles. Nanoparticles can also be formed using emulsion techniques as described below. An emulsion is a meta stable mixture of two immiscible liquids in which one of the liquids has been broken up into small droplets that are dispersed in the other liquid. An oil-in-water emulsion consists of small droplets of oil dispersed in a continuous water phase. Typically, the oil droplets have a surface-active agent associated with them to give the emulsion some stability. The size of the emulsion droplets can be from the micrometer to nanometer range. An emulsion in which the droplet size is less than about 200 nm (0.2 micron) is called a microemulsion. Oil-in-water emulsions can be made from almost any non water-soluble liquid.

Nanoparticles so formed can be associated with peptide drugs or protein drugs to stabilize the drugs against degradation and to enhance the absorption of large hydrophilic peptide or protein drugs. Nanoparticles with associated peptide or protein drugs for sublingual delivery should have an average diameter of between 1 nm and 1000 nm, preferably between 10 nm and 300 nm and most preferably between 20 nm and 200 nm. Nanoparticles with associated peptide or protein for oral delivery should have an average diameter of between 1 nm and 5000 nm, preferably between 200 nm and 3000 nm and most preferably between 500 nm and 2000 nm. Nanoparticles with associated peptide or protein for subcutaneous or intramuscular delivery should have an average diameter of between 1 nm and 1000 nm, preferably between 100 nm and 600 nm and most preferably between 200 nm and 500 nm. Nanoparticles with associated peptide or protein for intra-arterial or intravenous delivery should have an average diameter of between 1 nm and 500 nm, preferably between 10 nm and 300 nm and most preferably between 20 nm and 150 nm.

The size of the emulsion droplets can be controlled by several techniques. To obtain small droplets one may stir the oil in water mixture at very high speed or one may force the mixture to pass through a filter with very small pores by using a pressure differential. Depending upon the composition of the oil phase and the surfactants present one may obtain emulsions of almost any size droplet and with varying stability. Emulsion droplets may be used to form microparticles or nanoparticles. Solid materials that are dissolved in the oil droplets will solidify into particles of size of similar order of magnitude as the droplets upon removing the oil, which is acting as a solvent. The oil may be removed by extraction into another solvent in which the oil phase is soluble but the solute is not or by evaporation in the case of a volatile oil phase. These methods are typically used in the formation of microparticles and microspheres from polymeric materials such as polylactic-glycolic acid copolymers. The polymer material is dissolved in an organic non-water soluble solvent such as methylene chloride or ethyl acetate, an emulsion of the organic solution is made in the water phase (usually with a surfactant such as polyvinyl alcohol added) and the organic solvent is removed either by evaporation or by extraction leaving behind a suspension of microparticles or microspheres in the water phase. If a drug was included in the organic phase before the emulsification step then one obtains microspheres or microparticles containing a drug. If the emulsion was formed with small enough droplets then the size of the particles can be brought down into the nanometer range.

An alternate way of making micro or nanoparticles is to use an organic wax that is liquid above room temperature and solid at room temperature as the oil phase. A drug can be dissolved in the wax phase and the wax emulsified above room temperature. Upon cooling, one obtains a suspension of microparticles or nanoparticles of about the same size as the oil droplets in the emulsion without the need to extract or evaporate a solvent. Microparticles or nanoparticles of this sort are best suited for the delivery of oil soluble drugs and are best for forming long term delivery depots. The drug is embedded in an organic polymer or wax and is not immediately available for delivery until it can diffuse out of the matrix. Typically the diffusion takes place only after degradation of the particle matrix. Water-soluble drugs can be loaded into microparticles of this sort too, but in general with low drug loading and low efficiency. Since the water-soluble drug is not soluble in the oil phase, one needs to first make a water-in-oil emulsion which is then used to make the oil in water emulsion giving a water-in-oil in water double emulsion. For peptide and protein drugs where the solubility in water is not very high either, this drastically limits the amount of drug that can be incorporated into the system. Moreover, the drug is embedded within a non water-soluble matrix making it unavailable for immediate release as mentioned.

An improved system of nanoparticles or microparticles for more immediate use would have the peptide or protein drug attached to the microparticle or nanoparticle on the outer surface. Nanoparticles should be particularly useful for such a delivery system since their surface area per unit weight is great, allowing significant loading of drug. The down side of such a system was expected to be the lack of protection of the drug from degradative processes. We have surprisingly found, that when making nanoparticles from waxes that have surface active agents embedded in them and electrostatically binding a charged peptide or protein to the surface, the peptide is both available for delivery and protected against degradation.

In order to bond peptide or protein drugs to the surface of nanoparticles, the nanoparticle needs to be produced with a charged surface. This can be accomplished by making the nanoparticle from a polymer that contains ionic groups or by adding the charge by blending a charged molecule into the material making up the nanoparticle. In one embodiment of the invention waxes with melting points between 40 and 60 degrees are used. The waxes are melted and an ionic surfactant molecule is dissolved in the wax. The wax is then emulsified with warm (50-70 degrees Centigrade) water with the optional use of a non-ionic surfactant in the emulsification step. Suitable waxes for use in such a system are Witepsol® E85, microcrystalline wax, Stearic Acid, Compritol® 888 ATO, and Precirol® ATO 5 with Compritol® 888 ATO being a more preferable choice. Suitable anionic surfactant for such systems are sodium lauryl sulfate, sodium cholate, sodium taurocholate, and sodium ducosate with the ducosate being most preferred. When using sodium ducosate as the surface active agent it can be added in amounts between 1% and 30%, more preferably about 5% to 10% and most preferably around 7% by weight when compared to the wax.

The size of the molten wax droplets in the warm emulsion is controlled by the speed of the high shear mixer used to homogenize the mixture and can be determined by experimentation. The higher the speed and the longer the molten wax is homogenized in the water phase, the smaller the droplets will be. After the homogenization is complete, the mixture is cooled to below the solidification point of the wax. The nanoparticles thus formed have an embedded hydrophobic tail of the surfactant within them, thus immobilizing the surfactant with its charged head on the surface interacting with the aqueous phase.

Proteins or peptide drugs with a net positive charge can be attached electrostatically to the charged nanoparticles by mixing an aqueous solution of the peptide or protein with a suspension of the nanoparticles or microparticles. Examples of such proteins and peptides are glatiramer acetate, protamine sulfate, cationic antimicrobial peptides such as crecropins, magainin, diptericins, defensins, and gambicin, polylysine, polyarginine, and other peptides or proteins that have an excess total lysine plus arginine groups when compared to total aspartic acid plus glutamic acid groups. Cationic saccharide drugs such as gentamycin, amikacin and tobramycin can be used in place of the peptide or protein molecules.

Glatiramer acetate is the most preferred embodiment. The nano-suspension thus formed of peptide attached to the nanoparticles can be lyophilized to a powder for long-term storage. The lyophilized powder can be reconstituted in buffer to re-obtain the nano-suspension of drug. Nano-suspensions of attached drug thus obtained are particularly suited for oral delivery. If made with an average diameter below 200 nm the nano-suspension is suitable for sublingual delivery since nanoparticles can transverse the sublingual membrane. For oral delivery to the gastrointestinal tract larger nanoparticles are most preferred since they are the size most readily recognized by the Peyer's patches and M-cells. For such oral delivery the nano-suspension, as a lyophilized powder or as a reconstituted suspension, is to be delivered to the small intestine by using an enteric coated capsule. The enhanced stability of the peptide or protein when attached in the nano-suspension formulation allows for more time for the peptide drug to be absorbed in the intestine before it is degraded by enzymes in the gastrointestinal tract.

For bonding proteins and peptides with residual negative charge one needs to use a surface active agent in the preparation that has a cationic head group. Examples of such molecules are cetyltrimethylammonium bromide, chlorhexidine salts, hexadecyl triammonium bromide, dodecyl ammonium chloride and alkylpyridinium salts. The procedure for their formation is the same as when using surfactants with anionic charged heads. Examples of peptides and proteins with net negative charge are anionic anti microbial peptides such as enkelytin, peptide B, and dermicidin, polyaspartic acid, polyglutamic acid, and any peptide or protein that has a net excess of aspartic acid plus glutamic acid groups when compared to lysine plus arginine groups. Negatively charged polysaccharide drugs such as heparin or nucleic acid oligomers or polymers such as DNA or RNA and their fragments and antisense analogs of such, could also be used.

In a more preferred embodiment of this invention the wax used to form the nanoparticles is a wax formulated to spontaneously form a microemulsion. Such wax-with-surfactant formulations spontaneously form a microemulsion (droplet size less than 200 nm) when mixed with water above their melting point. The surfactant in such mixtures is often of the non ionic type so that an ionic surfactant need be added to the wax/surfactant formulation melt in order to obtain charged nanoparticles upon solidification of the microemulsion. Wax surfactant formulations that are suitable for our use are those that melt above room temperature and can be emulsified as a liquid in warm to hot water. Examples of such wax surfactant formulations are “emulsifying wax” which is a mixture of cetyl alcohol and polysorbate 60 in a molar ratio of about 20:1, polyoxyl 2 stearyl ether (Brij 72) and Tween 80 mixtures, and the Gelucire® series of waxes which are mixtures of 20% mono, di and tri glycerides, 72% mono and di fatty acid esters of PEG 1500 and 8% free PEG 1500. The Gelucire® series are preferred with Gelucire® 50/13 with a nominal melting point of 50° C. most preferred. Melted Gelucire® 50/13 spontaneously forms a clear microemulsion when mixed with water. In order to make the charged surface nanoparticles, it is necessary to add a charged surfactant to the wax melt to form the microemulsion with the surfactant groups incorporated. Suitable anionic surfactant for such systems are sodium lauryl sulfate, sodium cholate, sodium taurocholate, and sodium ducosate. Sodium ducosate is the most preferred surface-active agent with an anionic head. When using sodium ducosate as the surface active agent can be added in amounts between 1% and 30%, more preferably about 5% to 10% and most preferably around 7% by weight when compared to the wax formulation.

Proteins or peptide drugs with a net positive charge can be attached electrostatically to the charged nanoparticles thus formed by mixing an aqueous solution of the peptide or protein with a suspension of the nanoparticles or microparticles. Examples of such proteins and peptides are glatiramer acetate, protamine sulfate, cationic antimicrobial peptides such as crecropins, magainin, diptericins, defensins, and gambicin, polylysine, polyarginine, and other peptides or proteins that have an excess total lysine plus arginine groups when compared to total aspartic acid plus glutamic acid groups. Cationic saccharide drugs such as gentamycin, amikacin and tobramycin can be used in place of the peptide or protein molecules.

The nano-suspension thus formed of bound peptide to the nanoparticles can be lyophilized to a powder for long-term storage. The lyophilized powder can be reconstituted in buffer to re-obtain the nano-suspension of drug. Nano-suspensions of bound drug thus obtained are particularly suited for oral delivery as described above. If made with an average diameter below 200 nm the nano suspension is suitable for sublingual delivery since nanoparticles can transverse the sublingual membrane. These nano suspensions can also be used for oral delivery to the gastrointestinal tract for absorption through Peyer's patches and M-cells. For such oral delivery the nano-suspension, as a lyophilized powder or as a reconstituted suspension, is to be delivered to the small intestine by using an enteric coated capsule. The enhanced stability of the peptide or protein when bound in the nano-suspension formulation allows for more time for the peptide drug to be absorbed in the intestine before it is degraded by enzymes in the gastrointestinal tract.

In an embodiment 60-90% w/w Gelucire® 50/13 is used as the self emulsifying wax, 2-30% w/w sodium ducosate is used as the anionic surfactant, and 3-20% w/w glatiramer acetate is used as the peptide drug. In a most preferred embodiment about 80-85% w/w Gelucire® 50/13 is used as the self emulsifying wax, 5-7% w/w sodium ducosate is used as the anionic surfactant, and 8-15% w/w glatiramer acetate is used as the peptide drug. The nano suspension thus formed of bound peptide to the nanoparticles can be lyophilized to a powder for long term storage. The lyophilized powder can be reconstituted in buffer to re-obtain the nano suspension of drug.

Again here with the self emulsifying wax that forms a microemulsion for bonding proteins and peptides with residual negative charge one needs to use a surface active agent in the preparation that has a cationic head group. Examples of such molecules are cetyltrimethylammonium bromide, chlorhexidine salts, hexadecyl triammonium bromide, dodecyl ammonium chloride and alkylpyridinium salts. The procedure for their formation is the same as when using surfactants with anionic charged heads. Examples of peptides and proteins with net negative charge are anionic anti microbial peptides such as enkelytin, peptide B, and dermicidin, polyaspartic acid, polyglutamic acid, and any peptide or protein that has a net excess of aspartic acid plus glutamic acid groups when compared to lysine plus arginine groups. Negatively charged polysaccharide drugs such as heparin or nucleic acid oligomers or polymers such as DNA or RNA and their fragments and antisense analogs of such, could also be used.

Theraputic Uses

The present application contemplates that pharmaceutical compositions of nanoparticles with glatiramer acetate treat the same autoimmune diseases and inflammatory non-autoimmune diseases as glatiramer acetate (U.S. Patent Application Publication No. 20020055466 A1, published May 9, 2002 (Aharoni, et al.); U.S. Pat. No. 6,514,938 B1, issued Feb. 4, 2003 to Gad, et al.; PCT International Publication No. WO 01/60392, published Aug. 23, 2001 (Gilbert, et al.); U.S. Patent Application Publication No. 2004/0006022, published Jan. 8, 2004 (Strominger, et al.); U.S. Patent Application Publication No. 20020077278 A1, published Jun. 20, 2002 (Young, et al.); U.S. Patent Application Publication No. 20020037848 A1, published Mar. 28, 2002 (Eisenbach-Schwartz) and U.S. Patent Application Publication No. 20030004099 A1, published Jan. 2, 2003 (Eisenbach-Schwartz); PCT International Publication No. WO 01/97846, published Dec. 27, 2001 (Moses, et al.)).

### EXPERIMENTAL DETAILS

Materials

Gelucire® 50/13 is a commercially available semi-solid bioavailability enhancer and controlled-release agent for hard gelatin capsule formulations. Its chemical description is: Stearoyl macrogol-32 glycerides. Gelucire® 50/13 is synthesized by an alcoholysis/esterification reaction using hydrogenated palm oil and PEG 1500 as starting materials. Gelucire® 50/13 is therefore a well defined mixture of mono-, di-, and triglycerides and mono- and di-fatty acid esters of polyethylene glycol 1500, and free polyethylene glycol 1500. The predominant fatty acid is palmitostearic acid (C16-C18). Gelucire® 50/13 is a waxy solid (blocks or pellets), having a faint odor, a melting range (drop point) of 46.0 to 51.0° C., and a hydrophile-lipophile balance value of 13. European Pharmacopoeia 4rd edition: conforms to the “Stearoyl macrogolglycerides” monograph. U.S. Drug Master File no. 5253.

Docusate sodium, or sulfobutanediotic acid 1,4-bis-(2-ethylhexyl) ester sodium salt, is a pharmaceutical surfactant or wetting agent that has the formula C₂₀H₃₇NaO₇S and a molecular weight of 444.57 (The Merck Index, 12^{th} Ed.). Docusate sodium is commercially available, e.g. as Colace.

Glatiramer acetate (GA) is one example of a peptide drug for which the development of an oral alternative to delivery would be a significant advance. GA, an acetate salt of a synthetic copolymer of a random mixture of four amino acids, is one of the agents approved by the FDA for treating the relapsing-remitting form of Multiple Sclerosis (MS) (Physician's Desk Reference, 56 ed. pgs 3306-3310). MS is a chronic disease of the central nervous system that is characterized by inflammation, demyelination, and axon loss (van Ooston, B. W. et. al., Choosing Drug Therapy for Multiple Sclerosis. An Update., Drugs, 1998, 56(4), 555-569).

### Example 1

Gelucire® 50/13 wax (82.6 parts) was placed in a jacketed reactor fitted with a stirrer. The wax was melted by heating to about 70° C. with stirring. Sodium Ducosate (5.8 parts) was added and a solution of the ducosate in the melted wax was obtained. Preheated water (784 parts) was added and the mixture stirred at 200 rpm for 15 minutes at 70° C. A spontaneous microemulsion formed. The mass was then cooled to room temperature over a period of 120 minutes forming a nano suspension from the microemulsion. Glatiramer acetate (GA) (11.6 parts) was dissolved in 100 parts water and added to the stirred reactor. The mixture was stirred for thirty minutes allowing the GA to bind to the particles. The nano suspension was frozen at −20° C. for 12-20 hours and then lyophilized for 72 hours. A well formed cake was obtained. The lyophilized cake was milled in a QuadroComil milling machine through 0.8 mm screen to obtain a powder. The powder was reconstituted in phosphate buffer (0.05M pH 6.8) to obtain the drug nano suspension.

The particle size distribution of the nano suspension was measured using a Mastersizer2000, (Malvern Instruments Ltd., detection range 0.02-2000 um) by dispersing the particles in cold (14-20° C.) water. Results of the measurements were as follows:
d(0.1)=76 +/−5 nm
d(0.5)=124 +/−8 nm
d(0.9)=224 +/−4 nm

(Ten percent by volume [or weight] of the particles have a diameter less than 76 nm, 50% by volume [or weight] have a diameter less than 124 nm and 90% by volume [or weight] of the particles have a diameter of less than 224 nm).

The percent of the GA that is bound to the nanoparticles was determined by separating the free GA from the bound on a Sephadex column. The nanoparticles appear in the first fractions which correspond to the void volume of the column, while the free GA appears in later fractions after being retained on the column. To accomplish this separation, 25 mg of the reconstituted nanoparticles were loaded on a Sepahdex G-75 column (10×200 mm) and eluted with water at a flow rate of 0.5 ml/min. Fractions of 5 ml each were collected. Following the separation by UV absorption at 275 nm indicated that there were two peaks of material, one at the void volume corresponding to the first 7 fractions of the eluate and the other in subsequent fractions. The two peaks were well separated. The amount of GA in each fraction was determined by an HPLC analysis on a Superose 12 HR 10/30 column (Pharmacia, 10×30 mm) using an acidic phosphate buffer (0.1 M, pH=1.5) at a flow of 0.5 ml/min and using UV detection at 208 nm. The acidic buffer removes the GA from the particles and allows the determination of total GA in the fraction. The percent of GA bound in the sample was the ratio of the total amount of GA determined in the fractions corresponding to the void volume peak, to the total amount of GA determined in all the fractions multiplied by 100. The percent bound was found to be 50 +/−5% (w/w).

### Example 2

Gelucire® 50/13 wax (72.2 parts) was placed in a jacketed reactor fitted with a stirrer. The wax was melted by heating to about 70° C. with stirring. Sodium Ducosate (5.4 parts) was added and a solution of the ducosate in the melted wax was obtained. Preheated water (684 parts) was added and the mixture stirred at 200 rpm for 15 minutes at 70° C. A spontaneous microemulsion forms. The mass was then cooled to room temperature over a period of 120 minutes forming a nano suspension from the microemulsion. Glatiramer acetate (GA) (10.9 parts) was dissolved in 100 parts water and added to the stirred reactor. The mixture was stirred for thirty minutes allowing the GA to bind to the particles. Polyvinylpyrrolidone (PVP k30, 11.5 parts) was dissolved in 100 parts water and added to the nano suspension. The nano suspension was frozen at −20° C. for 12-20 hours and then lyophilized for 72 hours.

A well formed cake was obtained. The lyophilized cake was milled in a Quadro Comil milling machine through 0.8 mm screen to obtain a powder. The powder was reconstituted in phosphate buffer (0.05M pH=6.8) to obtain the drug nano suspension.

The particle size distribution was measured as in Example 1 and found to be very similar i.e.
d(0.1)=79 +/−2 nm
d(0.5)=121 +/−1 nm
d(0.9)=250 +/−16 nm
   and the percent bound was determined as in Example 1 but with the UV detection in the GA content analysis being at 280 nm and found to be 50 +/−5% (w/w) as before.

### Example 3

### Protection from Enzymatic Degradation

The enzymatic degradation of free GA in solution versus GA in the nano suspension (˜50% bound to the nanoparticles) and versus GA bound to the nanoparticles (˜100% bound) was studied using pancreatin. The GA bound to the nanoparticles was prepared by collecting fractions from the void volume of the Sephadex column as described in Example 1 and pooling the samples. Pancreatin is a mixture of pancreatic proteases consisting of trypsin and chymotrypsins.

Pancreatin 3.5 mg was dissolved in 10 ml of 0.05M phosphate buffer pH=6.8. Free GA, GA in nano suspension, or fully bound GA, 35 mg as GA, was dissolved or suspended in 10 ml of 0.05M phosphate buffer pH=6.8. 1 ml of the pancreatin solution and 1 ml of the GA solution or suspension were mixed (the ratio of pancreatin to GA was fixed at 1:10) and held at 37° C. At fixed time points the enzymatic reaction was stopped by adding 0.4 ml of 1N HCl. The residual GA content of the mixture was determined by the HPLC method described in Example 1. The results of the percent residual GA at various time points is given in the table 1.

TABLE 1

| | | Nano | | Nanoparticle |
|---|---|---|---|---|
| | | suspension | Nano | Bound |
| | | (˜50% | suspension | GA only |
| Time | | bound to | (˜50% bound to | (˜100% |
| [min] | Free GA | particles) | particles) + PVP | bound) |
| 0 | 100.0 +/− 0.0 | 100.0 +/− 0.0 | 100.0 | 100.0 |
| 1 | 75.1 +/− 3.9 | 87.7 +/− 4.1 | 90.4 | 98.8 |
| 2 | 63.4 +/− 4.8 | 83.0 +/− 2.8 | 81.2 | 94.9 |
| 3 | 56.3 +/− 4.7 | 80.8 +/− 4.1 | 78.6 | 91.2 |
| 6 | 47.1 +/− 4.4 | 74.6 +/− 7.3 | | 80.0 |
| 10 | | 66.5 +/− 5.9 | | 74.5 |
| 15 | | 56.4 +/− 4.0 | | |

After three minutes there was 56% of the free GA remaining while the nano suspension showed 81% remaining. The fully bound GA showed 91% remaining. The nano suspension showed 56% remaining after 15 minutes or 5 times as long as the free GA. The enhanced stability of the GA in the nano suspension formulation should aid in its ability to be absorbed in various parts of the gastrointestinal tract.

GA has been shown to significantly reduce the relapse rate of multiple sclerosis and to significantly reduce the burden of disease lesions as seen in magnetic resonance imaging measures (Simpson, D. et. al. Glatiramer Acetate: a Review of its use in Relapsing-Remitting Multiple Sclerosis, CNS— Drugs, 2002, 16(12), 825-850). GA is an acetate salt of a synthetic copolymer of a random mixture of four amino acids (Physician's Desk Reference, 56 ed. pgs 3306-3310) and while its mechanism of action has not been totally elucidated, it is believed to down-regulate pro-inflammatory cytokines, up-regulate anti-inflammatory cytokines, and to interfere with antigen presentation (Zhang, J. et. al., A Comparison of Action of Interferon beta and Glatiramer Acetate in the Treatment of Multiple Sclerosis, Clin. Ther. 2002, 24(12), 1998-2021; Miller, A. et. al. Treatment of Multiple Sclerosis with Copolymer-1 (Copaxone): Implicating Mechanisms of Th1 to Th2/Th3 Immune Deviation, J. Neurimmunol., 1998, 92(1-2), 113-121; Ragheb, S. et. al., Long-Term Therapy with Glatiramer Acetate in Multiple Sclerosis: Effect on T-cells, Mult. Scler. 2001, 7(1), 43-47; Hussien, Y et. al., Glatiramer Acetate and IFN-beta Act on Dendritic Cells in Multiple Sclerosis, J. Neuroimmunol., 2001, 121(1-2), 102-110) leading to less destruction of axonal myelin. GA, administered as a 20 mg subcutaneous daily injection, has shown extended clinical benefits (Simpson, D. et. al. Glatiramer Acetate: a Review of its use in Relapsing-Remitting Multiple Sclerosis, CNS— Drugs, (2002), 16(12), 825-850; Johnson, K. P., et. al., Sustained Clinical Benefits of Glatiramer Acetate in Relapsing Multiple Sclerosis patients Observed for Six Years. Copolymer-1 Multiple Sclerosis Study Group, Mult. Scler. (2000), 6(4), 255-266) for MS patients. GA has a more benign side effect profile than other immunomodulatory agents that are approved for use and is very well tolerated (Simpson, D. et. al. Glatiramer Acetate: a Review of its use in Relapsing-Remitting Multiple Sclerosis, CNS— Drugs, (2002), 16(12), 825-850; Francis, D. A., Glatiramer Acetate, Int. J. Clin. Pract. 2001, 55(6), 394-398). GA, as well as the other agents, are protein or peptide drugs and are administered by injection only. The development of an oral alternative to delivery of this drug is a significant improvement in therapy due to patient convenience and preference.

To solve the problem of oral delivery of peptide and protein drugs to a human it was necessary to find methods that can stabilize the drug against degradation in the gastrointestinal tract and at the same time enhance the absorption of the large hydrophilic molecules. We have surprisingly found that nanoparticles with a surface attached peptide or protein drug can carry out both of these functions.

### Example 4

### Efficacy in Rat Experimental Autoimmune Encephalomyelitis (EAE)

Experimental autoimmune encephalomyelitis (also called experimental allergic encephalomyelitis or EAE) is a demyelinating disease of the central nervous system that mimics multiple sclerosis (MS) (Zamvil, S. S., Steinman, L. The T lymphocyte in experimental allergic encephalomyelitis, Annu Rev Immunol 1990, 8, 579-621). It is induced in rats or mice by immunizing them with myelin antigens and various toxins in media such as Freund's adjuvant. (Bernard, C. C., Carnegie, P. R.), Experimental autoimmune encephalomyelitis in mice: immunologic response to mouse spinal cord and myelin basic proteins, J. Immunol. 1975 May; 114(5): 1537-40; Beck, F. W., Whitehouse, M. W., Pearson, C. M., Improvements for consistently inducing experimental allergic encephalomyelitis (EAE) in rats: I. without using mycobacterium. II. inoculating encephalitogen into the ear, Proc. Soc. Exp. Biol. Med. 1976 March; 151(3): 615-22) and is used as a model for testing the efficacy of drugs in treating MS.

In this experiment the goal was to reduce experimental autoimmune encephalomyelitis (EAE) induced clinical signs in the Lewis rat model. Lewis (LEW) rats are highly susceptible to EAE induced with guinea pig spinal cord homogenate (GPSCH) emulsified in modified complete Freund's adjuvant (CFA). The earliest clinical signs begin 10-12 days after immunization and most rats recover by 18 to 20 days. Decreased signs of EAE in rats with test materials administered orally during the study is considered a protective effect set forth by the experimental design.

The animal were induced with the disease by subcutaneous injection of GPSCH emulsion into both hind paws on day 1.

Initiation of scoring of rats for EAE clinical signs started on day 9. The treatment groups were blinded to the scorer. The treatments or control treatments were started on day 1 and administered daily to day 22. Treatments were administered by oral gavage at a dose and frequency described in Table 2.

There were ten rats in each group.

TABLE 2
Treatments

| Treatment group | Dose | Frequency |
|---|---|---|
| Double Distilled | 5 ml/kg | Daily gavage |
| Water (DDW) - | | starting on |
| control | | day 1 |
| Wax-PVP | (21 mg | Daily gavage |
| nanoparticles - | particles)/ml | starting on |
| control | 5 ml/kg | day 1 |
| Glatiramer | (2.5 mg GA)/ml | Daily gavage |
| Acetate | 5 ml/kg | starting on |
| solution | | day 1 |
| Glatiramer | (2.5 mg GA bound | Daily gavage |
| Acetate - Wax - | to 21 mg | starting on |
| PVP | particles)/ml | day 1 |
| nanoparticles | 5 ml/kg | |

The animals were accessed daily for EAE clinical signs as described in Table 3.

TABLE 3
Evaluation of the EAE Clinical Signs.

| Score | Clinical Signs | Description |
|---|---|---|
| 0 | Normal behavior | No clinical signs |
| 1 | Tail weakness | The tail is limp and |
| | | droops. |
| 2 | Hind legs hypotonia and | Limb pareses, wobbly |
| | weakness. | walk- when the rat |
| | | walks the hind legs |
| | | are unsteady or it |
| | | drags one hind leg. |
| 3 | Hind legs paralysis, | The rat can't move its |
| | front legs normal | hind legs and it drags |
| | | them when it walks. |
| | | Forelegs are normal |
| 4 | Hind legs paralysis, | The rat can't move its |
| | front legs weak | hind legs and it drags |
| | | them when it walks. |
| | | Forelegs are weak |
| 5 | Full paralysis | The rat can't move all |
| | | its four legs at all. |
| 6 | Moribund animals/Death. | |

The following parameters were calculated:

Calculation of the Incidence, Mortality, Onset and Duration of Disease

The number of sick animals in each group were assessed. The day of onset of the symptoms, the duration of days of the symptoms and the number of animals dying were noted and the percentages calculated.

Calculation of the Mean Maximal Score (MMS)

The maximal scores of each of the animals in each group were summed. The mean maximal score of the group was calculated as follows:

Σ Maximal score of each rat/number of rats in the group.

Calculation of the Group Mean Score (GMS)

The scores of each of the animals in the group were summed and the mean score per day was calculated. The group mean score was calculated as follows:
Σ Total score of each rat per day/number of rats in the group.
   Results

Table 4 presents the data for disease incidence in the test animals.

TABLE 4
Incidence of EAE and Mortality

| | |
|---|---|
| Incidence | Mortality |
| Total | Total |

| Group | N | % | N | % |
|---|---|---|---|---|
| DDW | 10/10 | 100 | 1/10 | 10 |
| GA | 8/9 | 89 | 0/10 | 0 |
| WAX-PVP | 10/10 | 100 | 0/10 | 0 |
| GA-WAX-PVP | 9/10 | 90 | 0/10 | 0 |

The EAE was induced in all of the test animals in the water treatment control group and the wax-PVP nanoparticle vehicle control group while the disease was induced in eight of nine animals in the Glatiramer acetate oral solution group and nine of ten in the Glatiramer acetate nanoparticle treatment group. Only one animal died in the study.

Table 5 presents the duration and onset of the disease.

TABLE 5
EAE Duration and Onset

| | Mean | |
|---|---|---|
| | Disease | Mean Onset |
| Group | Duration, days | days |
| DDW | 4.6 ± 1.4 | 11.9 ± 1.0 |
| GA | 3.2 ± 1.6 | 13.0 ± 2.5 |
| WAX-PVP | 4.2 ± 0.8 | 11.8 ± 0.8 |
| GA-WAX-PVP | 3.4 ± 1.6 | 13.1 ± 2.3 |

The mean duration of the disease was shorter for the two Glatiramer acetate groups (3.2 and 3.4 days) than for the two control groups (4.6 and 4.2 days). The mean days to onset of disease symptoms was delayed in the two GA groups (13.0 and 13.1 days) when compared to the two vehicle groups (11.9 and 11.8 days). The Glatiramer acetate nanoparticle treatment group showed a slightly longer delay in onset but also a slightly longer duration of disease when compared to the oral solution Glatiramer acetate group. These differences were probably not significant.

Table 6 collects the mean daily scores for each of the groups and serves as a measure of disease severity.

TABLE 6
Daily Mean Score

Mean score

| | | | Wax- | |
|---|---|---|---|---|
| days | DDW | GA | PVP | GA-Wax-PVP |
| 9 | 0.0 | 0.0 | 0.0 | 0.0 |
| 10 | 0.0 | 0.0 | 0.0 | 0.0 |
| 11 | 0.8 | 0.2 | 0.7 | 0.2 |
| 12 | 1.1 | 1.3 | 1.4 | 0.8 |
| 13 | 2.1 | 2.0 | 2.1 | 1.3 |
| 14 | 2.5 | 1.8 | 1.9 | 1.6 |
| 15 | 2.1 | 1.2 | 1.1 | 1.3 |
| 16 | 1.0 | 0.1 | 0.4 | 0.3 |
| 17 | 0.6 | 0.0 | 0.0 | 0.0 |
| 18 | 0.6 | 0.0 | 0.0 | 0.0 |
| AUC | 10.8 | 6.6 | 7.6 | 5.5 |

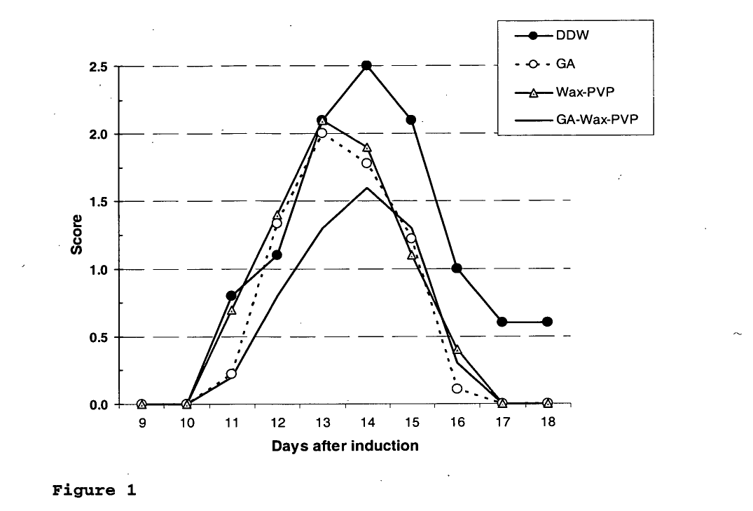

The symptoms started to manifest themselves on day 11 and peaked at day 13 or 14. The mean severity of the disease was highest with the treatment with the vehicles alone reaching 2.5 on day 14 with the water treatment and 2.1 at day 13 with the wax-PVP nanoparticle vehicle. Oral glatiramer acetate was somewhat better than the wax-PVP vehicle while the glatiramer acetate conjugated to the wax-PVP (GA-wax-PVP) gave considerably improved results reaching a maximum of only 1.6 on day 14. The area under the curve (AUC) shows that the GA-wax-PVP has a total severity of about half that of the water negative control and lower than the oral administration of GA not conjugated to the nanoparticles. The oral GA results are better than either vehicle treatment group. These results are shown graphically in FIG. 1. The mean group scores are presented in Table 7.

TABLE 7
Mean Scores and % Inhibition

| | Mean | Group | % of | |
|---|---|---|---|---|
| | Maximal | Mean | inhibition | |
| Group | Score | Score | vs. its placebo | vs. DDW |
| DDW | 2.7 ± 1.3 | 1.08 ± 1.0 | | |
| GA | 2.2 ± 1.1 | 0.67 ± 0.4 | 38% | 38% |
| WAX-PVP | 2.4 ± 1.0 | 0.76 ± 0.3 | | |
| GA-WAX-PVP | 1.9 ± 0.9 | 0.55 ± 0.3 | 28% | 49% |

As in the previous tables the data presented here shows that mean score for each treatment group was better for the two glatiramer acetate treatments (0.67 and 0.55) than for the two vehicle control treatments (1.08 and 0.76) with the glatiramer acetate nanoparticles showing the best results. The mean of the maximal disease severity in each group followed the same tend with the glatiramer acetate nanoparticles having the lowest mean score (1.9). The glatiramer acetate oral solution showed a 38% inhibition of group mean score when compared to DDW, its control treatment. The glatiramer acetate nanoparticles showed a 28% inhibition in the mean group score compared to its vehicle as a control and a 49% inhibition when compared to DDW as a control.

### Conclusion

The Example shows that the administration of an oral solution of glatiramer acetate or the oral administration of the glatiramer acetate bound to nanoparticles inhibit the disease severity of EAE in rats. Treatment with the nanoparticle bound GA was shown to be more efficacious in most of the disease severity parameters.

### Example 5

### Determination of Immunologic Activity of GA-Wax-PVP

The ability of GA-Wax-PVP to elicit an immune response with a similar cytokine pattern as glatiramer acetate solution was tested in an ex-vivo model in mice. The study was conducted over a period of 10 days. Mice were fed daily with GA reference standard (GA RS, 250 μg), GA-Wax-PVP (amount equivalent to 250 μg GA) or Wax-PVP as negative control (similar volume and weight of particles as GA-Wax-PVP). During the study period, subsets of animals in each treatment group were sacrificed on days 3, 6 and 10. Spleens were excised and primary cell cultures were prepared. The effect of the treatment was tested by in-vitro activation of splenocytes with GA RS. The response of the cells to the challenge is a measure of previous exposure to GA. Comparing the immune response of the mice fed GA-Wax-PVP to those fed GA RS gives a measure of the relative exposure to GA that each treatment provided. T-cell response was monitored by detection of cytokines secreted from activated cells by ELISA analysis. The levels of IL-2, TGF-β, IL-4, IL-5, IL-10 and IFN-γ were examined.

Results

The levels of all the cytokines examined throughout the test period were similar for the mice, fed GA RS and those fed GA-Wax-PVP.

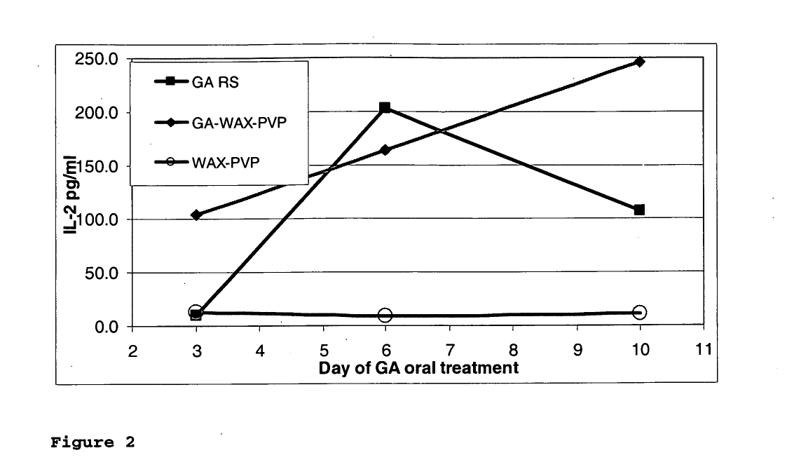

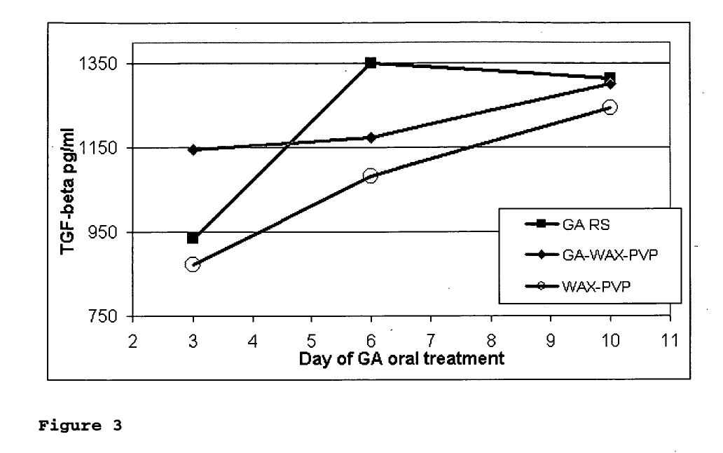

The results of IL-2 and TGF-β measurements are shown below. FIG. 2 shows that the Wax-PVP did not elicit any IL-2 response while the GA-Wax-PVP elicited a response that is similar in magnitude to the GA RS. FIG. 3 also shows that the magnitude of the secretion of TGF-β to the GA RS challenge is again of similar magnitude whether the mice were treated with GA RS or GA-Wax-PVP. The negative control also elicited a response, which was smaller than the response elicited with the test substance, with this marker.

### Conclusion

The GA-Wax-PVP particles are immunologically active and the cytokine pattern for GA-WAX-PVP and GA RS are similar.

1. A pharmaceutical composition comprising a nanoparticle and any one of a peptide, a polysaccharide, or a glycoprotein, attached electrostatically thereto, and a pharmaceutically acceptable carrier.

2. The pharmaceutical composition of claim 1, wherein the nanoparticle comprises an organic wax having a melting point from 40° C. to 60° C.

3. The pharmaceutical composition of claim 2, wherein the organic wax is stearic acid; atomized glyceryl palmitostearate; atomized glyceryl behenate; a paraffin wax having a melting point from 40° C. to 60° C.; a mixture of mono-, di-, and tri-glycerides obtained by esterification of fatty acids of natural origin with glycerol and having a melting point from 40° C. to 60° C.; a mixture of mono-, di-, and tri-glycerides obtained by transesterification of fatty acids of natural origin and having a melting point from 40° C. to 60° C. or a mixture of mono-, di-, and tri-glycerides of C12-C18 fatty acids and having a melting point from 40° C. to 60° C.

4. The pharmaceutical composition of claim 2, wherein the organic wax is admixed with a nonionic surfactant.

5. The pharmaceutical composition of claim 4, wherein the admixture of organic wax and non-ionic surfactant is cetyl alcohol with polysorbate 60, polyoxyl 2 stearyl ether with polysorbate 80, or mono-, di-, and tri-glycerides with free polyethylene glycol and with mono-, and di-fatty acid esters of polyethylene glycol.

6. The pharmaceutical composition of claim 1, wherein the nanoparticle comprises an ionic surface-active agent.

7. The pharmaceutical composition of claim 6, wherein the surface-active agent has a charged head and a hydrophobic tail.

8. The pharmaceutical composition of claim 7, wherein the surface-active agent is an anionic surfactant.

9. The pharmaceutical composition of claim 8, wherein the anionic surfactant is sodium lauryl sulfate, sodium cholate, sodium taurocholate, or sodium docusate.

10. The pharmaceutical composition of claim 9, wherein the surface-active agent is sodium docusate.

11. The pharmaceutical composition of claim 1, wherein the peptide, the polysaccharide, or the glycoprotein has a net positive charge and the nanoparticle has a net negative charge, such that the peptide, the polysaccharide, or the glycoprotein is electrostatically attached to the nanoparticle.

12. The pharmaceutical composition of claim 11, wherein the peptide is attached to the nanoparticle, the peptide having more combined total lysine plus arginine groups than the combined total of aspartic acid plus glutamic acid groups.

13. The pharmaceutical composition of claim 11, wherein the peptide is glatiramer acetate.

14. The pharmaceutical composition of claim 11, wherein the peptide is interferon.

15. The pharmaceutical composition of claim 11, wherein the polysaccharide is attached to the nanoparticle, the polysaccharide being gentamycin, amikacin or tobramysin.

16. The pharmaceutical composition of claim 7, wherein the surface-active agent is a cationic surfactant.

17. The pharmaceutical composition of claim 16, wherein the cationic surfactant is cetyltrimethylammonium bromide, chlorhexidine salts, hexadecyl triammonium bromide, dodecyl ammonium chloride or an alkylpyridinium salt.

18. The pharmaceutical composition of claim 1, wherein the peptide, the polysaccharide, or the glycoprotein has a net negative charge and the nanoparticle has a net positive charge, such that the peptide, the polysacharide, or the glycoprotein is electrostatically attached to the nanoparticle.

19. The pharmaceutical composition of claim 18, wherein the peptide is attached to the nanoparticle, the peptide having more combined total aspartic acid plus glutamic acid groups than lysine plus arginine groups.

20. The pharmaceutical composition of claim 18, wherein the polysaccharide is attached to the nanoparticle, the polysaccharide being heparin.

21. The pharmaceutical composition of claim 1, characterized in that the rate of enzymatic degradation of the peptide, the polysaccharide, or the glycoprotein when electrostatically attached to the nanoparticle is lower than the rate of enzymatic degradation of the peptide, the polysaccharide, or the glycoprotein when unattached to the nanoparticle in solution.

22. A pharmaceutical composition comprising nanoparticles of i) an admixture of mono-, di-, and tri-glycerides with free polyethylene glycol and with mono-, and di-fatty acid esters of polyethylene glycol, ii) sodium docusate, and iii) glatiramer acetate.

23. The pharmaceutical composition of claim 22, wherein the admixture comprises 60-90% by weight of the composition, wherein the sodium docusate comprises 2-30% by weight of the composition, and wherein the glatiramer acetate comprises 3-20% of the composition.

24. The pharmaceutical composition of claim 23, wherein the admixture comprises 80-85% by weight of the composition, wherein the sodium docusate comprises 5-7% by weight of the composition, and wherein the glatiramer acetate comprises 8-15% of the composition.

25. The pharmaceutical composition of claim 22, wherein the admixture comprises 20% mono-, di-, and tri-glycerides, 8% free polyethylene glycol, and 72% mono-, and di-fatty acid esters of polyethylene glycol.

26. The pharmaceutical composition of claim 22, characterized in that the rate of enzymatic degradation of the glatiramer acetate when electrostatically attached to the nanoparticle is lower than the rate of enzymatic degradation of the glatiramer acetate when unattached to the nanoparticle in solution.

27. The pharmaceutical composition of claim 22, wherein the nanoparticle has an average diameter of between 1 nm and 5000 nm.

28. The pharmaceutical composition of claim 27, wherein the nanoparticle has an average diameter of between 200=m and 3000 nm.

29. The pharmaceutical composition of claim 28, wherein the nanoparticle has an average diameter of between 500 nm and 2000 nm.

30. The pharmaceutical composition of claim 27, wherein the nanoparticle has an average diameter of between 1 nm and 1000 nm.

31. The pharmaceutical composition of claim 30, wherein the nanoparticle has an average diameter of between 1 nm and 500 nm.

32. The pharmaceutical composition of claim 31, wherein the nanoparticle has an average diameter of between 10 nm and 300 nm.

33. The pharmaceutical composition of claim 32, wherein the nanoparticle has an average diameter of between 20 nm and 200 nm.

34. The pharmaceutical composition of claim 33, wherein the nanoparticle has an average diameter of between 20 nm and 150 nm.

35. The pharmaceutical composition of claim 30, wherein the nanoparticle has an average diameter of between 100 nm and 600 nm.

36. The pharmaceutical composition of claim 35, wherein the nanoparticle has an average diameter of between 200 nm and 500 nm.

37. A lyophilized pharmaceutical composition of claim 1.

38. A process for preparing the pharmaceutical composition of claim 1 comprising
i) forming a spontaneous microemulsion by heating to above 50° C. a mixture of water, and a wax;
ii) cooling the microemulsion to room temperature to form nanoparticles; and
iii) contacting the nanoparticels with the peptide, the polysaccharide, or the glycoprotein to form the pharmaceutical composition.

39. The process of claim 38, wherein the wax is an organic wax having a melting point from 40° C. to 60° C.

40. The process of claim 39, wherein the organic wax is stearic acid; atomized glyceryl palmitostearate; atomized glyceryl behenate; paraffin waxes having a melting point from 40° C. to 60° C.; a mixture of mono-, di-, and tri-glycerides obtained by esterification of fatty acids of natural origin with glycerol and having a melting point from 40° C. to 60° C.; a mixture of mono-, di-, and tri-glycerides obtained by transesterification of fatty acids of natural origin and having a melting point from 40° C. to 60° C.; and a mixture of mono-, di-, and tri-glycerides of C12-C18 fatty acids and having a melting point from 40° C. to 60° C.

41. The process of claim 38, wherein the wax comprises a non-ionic surfactant.

42. The process of claim 38, wherein the wax comprises an ionic surfactant.

43. The process of claim 41, wherein the wax with non-ionic surfactant is cetyl alcohol with polysorbate 60, polyoxyl 2 stearyl ether with polysorbate 80, or mono-, di-, and tri-glycerides with free polyethylene glycol and with mono-, and di-fatty acid esters of polyethylene glycol.

44. The process of claim 42, wherein the ionic surfactant is an anionic surfactant.

45. The process of claim 44, wherein the anionic surfactant is sodium lauryl sulfate, sodium cholate, sodium taurocholate, or sodium docusate.

46. The process of claim 45, wherein the anionic surfactant is sodium docusate.

47. The process of claim 42, wherein the ionic surfactant is a cationic surfactant.

48. The process of claim 47, wherein the cationic surfactant is cetyltrimethylammonium bromide, chlorhexidine salts, hexadecyl triammonium bromide, dodecyl ammonium chloride or an alkylpyridinium salt.

49. The process of claim 38, wherein the peptide, the polysaccharide, or the glycoprotein has a net positive charge and the nanoparticle has a net negative charge, such that the peptide, the polysaccharide, or the glycoprotein is electrostatically attached to the nanoparticle.

50. The process of claim 49, wherein the peptide is attached to the nanoparticle, the peptide having more combined total lysine plus arginine groups than the combined total of aspartic acid plus glutamic acid groups.

51. The process of claim 49, wherein the polysaccharide is attached to the nanoparticle, the polysaccharide being gentamycin, amikacin or tobramysin.

52. The process of claim 49, wherein the peptide is glatiramer acetate.

53. The process of claim 49, wherein the peptide is interferon.

54. The process of claim 38, wherein the peptide, the polysaccharide, or the glycoprotein has a net negative charge and the nanoparticle has a net positive charge, such that the peptide, the polysacharide, or the glycoprotein is electrostatically attached to the nanoparticle.

55. The process of claim 54, wherein the peptide is attached to the nanoparticle, the peptide having more combined total aspartic acid plus glutamic acid groups than lysine plus arginine groups.

56. The process of claim 54, wherein the polysaccharide is attached to the nanoparticle, the polysaccharide being heparin.

57. A method of delivering to a subject a peptide, a polysaccharide, or a glycoprotein, comprising administering to the subject the pharmaceutical composition of claim 1.

58. The method of claim 57, wherein the administration is sublingual, orally to the stomach, orally to the small intestine, orally to the large intestine, intramuscular, subcutaneous, intra-arterial or intravenous.

59. A method of inhibiting enzymatic degradation of a peptide, a polysaccharide, or a glycoprotein upon oral ingestion of the peptide, the polysaccharide, or the glycoprotein by an animal, comprising electrostatically attaching the peptide, the polysaccharide, or the glycoprotein to a nanoparticle prior to the oral ingestion, so as to thereby inhibit enzymatic degradation of the peptide, the polysaccharide, or the glycoprotein upon oral ingestion.

60. The method of claim 59, wherein the peptide is glatiramer acetate.

61. The method of claim 59, wherein the nanoparticle comprises i) an admixture of mono-, di-, and tri-glycerides with free polyethylene glycol and with mono- and di-fatty acid esters of polyethylene glycol, and ii) sodium docusate.

62. The method of claim 59, wherein the step of electrostatically attaching the peptide, the polysaccharide, or the glycoprotein to the nanoparticle comprises
i) forming a spontaneous microemulsion by heating to above 50° C. a mixture of water, and a wax;
ii) cooling the microemulsion to room temperature to form nanoparticles; and
iii) contacting the nanoparticles with the peptide, the polysaccharide, or the glycoprotein, thereby electrostatically attaching the peptide, the polysaccharide, or the glycoprotein to the nanoparticle.

63. A method of delivering to a subject a deoxyribonucleic acid molecule or a ribonucleic acid molecule, comprising administering to the subject a pharmaceutical composition comprising
the deoxyribonucleic acid molecule or the ribonucleic acid molecule attached electrostatically to a nanoparticle, and
a pharmaceutically acceptable carrier, wherein the administration is oral or sublingual.

64. The method of claim 63, wherein the nanoparticle comprises an organic wax having a melting point from 40° C. to 60° C.

65. The method of claim 64, wherein the organic wax is stearic acid; atomized glyceryl palmitostearate; atomized glyceryl behenate; a paraffin wax having a melting point from 40° C. to 60° C.; a mixture of mono-, di-, and tri-glycerides obtained by esterification of fatty acids of natural origin with glycerol and having a melting point from 40° C. to 60° C.; a mixture of mono-, di-, and tri-glycerides obtained by transesterification of fatty acids of natural origin and having a melting point from 40° C. to 60° C.; and a mixture of mono-, di-, and tri-glycerides of C12-C18 fatty acids and having a melting point from 40° C. to 60° C.

66. The method of claim 64, wherein the organic wax is admixed with a non-ionic surfactant.

67. The method of claim 66, wherein the admixture of organic wax and non-ionic surfactant is cetyl alcohol with polysorbate 60, polyoxyl 2 stearyl ether with polysorbate 80, or mono-, di-, and tri-glycerides with free polyethylene glycol and with mono-, and di-fatty acid esters of polyethylene glycol.

68. The method of claim 63, wherein the nanoparticle comprises a cationic surfactant.

69. The method of claim 68, wherein the cationic surfactant is cetyltrimethylammonium bromide, chlorhexidine salts, hexadecyl triammonium bromide, dodecyl ammonium chloride or an alkylpyridinium salt.

70. A pharmaceutical composition prepared by the process claim 38.

71. A pharmaceutical composition prepared by the process of claim 52.

72. The pharmaceutical composition of claim 13 comprising glatiramer acetate in an amount effective to treat an autoimmune disease or an inflammatory non-autoimmune disease in a subject, and a pharmaceutically acceptable carrier.

73. The pharmaceutical composition claim 13 comprising glatiramer acetate in an amount effective to treat multiple sclerosis in a subject, and a pharmaceutically acceptable carrier.

74. A method for treating a subject afflicted with an autoimmune disease or an inflammatory non-autoimmune disease, which comprises administering to the subject the pharmaceutical composition of claim 72.

75. A method for treating a subject afflicted with relapsing remitting multiple sclerosis which comprises administering to the subject the pharmaceutical composition of claim 73.

76. The method of claim 75, wherein the administration is through intravenous, intraperitoneal, intramuscular, subcutaneous, oral, intranasal, buccal, vaginal, rectal, intraocular, intrathecal, topical, sublingual or intradermal routes.

77. The method of claim 76, wherein the administration is oral.

78. A method of treating a subject afflicted with relapsing remitting multiple sclerosis which comprises oral administration of a nanoparticulate formulation of glatiramer acetate, wherein the amount of glatiramer acetate in the nanoparticulate formulation is effective to alleviate a symptom of the relapsing-remitting multiple sclerosis in the subject.
